(19) United States
(12) Patent Application Publication
(10) Pub. No.: US 2007/0185389 A1
(43) Pub. Date: Aug. 9, 2007

(54) SYSTEMS AND METHODS FOR REAL TIME DIAGNOSTIC AND DATA CORRELATION UTILIZING A COMPUTER NETWORK

(76) Inventor: Robin Peng, South Jordan, UT (US)

Correspondence Address: BAKER & ASSOCIATES PLLC, 470 EAST NINTH AVENUE, SALT LAKE CITY, UT, 84103, US

(21) Appl. No.: 11/670,980

(22) Filed: Feb. 3, 2007

Related U.S. Application Data
(60) Provisional application No. 60/765,269, filed on Feb. 4, 2006.

(51) Int. Cl. A61B 5/00; G06Q 10/00

(52) U.S. Cl. 600/300; 705/2

(57) ABSTRACT

The present invention relates to systems and methods for real time diagnostic and data correlation utilizing a computer network. One embodiment of the present invention relates to a method for interfacing a health monitoring system with an on-line medical information site to provide real-time diagnostics and or updated research data correlation. The method includes measuring and recording at least two health related values, compiling the at least two health related values into a format consistent with an online medical information database, and transmitting the compiled versions of the at least two health related values to an online medical information database. The method may further include receiving real time diagnostics from a medical professional. The method may also include receiving updated medical research data corresponding to the transmitted values. An alternative embodiment of the present invention relates to a system for providing real time remote diagnostic and correlated research data. The system includes a medical monitoring system configured to measure at least two health related values, a remote medical database which is accessible by diagnostic medical professionals and which contains updated medical research data, and a communication link between the medical monitoring system and the remote medical database.

### CROSS-REFERENCES TO RELATED APPLICATIONS

The present invention claims priority on earlier U.S. Provisional Application 60/765,269, filed Feb. 4, 2006, and incorporates said earlier Application herein by reference in its entirety.

### FIELD OF THE INVENTION

The invention generally relates to health care. In particular, the present invention relates to systems and methods for real time diagnostic and data correlation utilizing a computer network.

### BACKGROUND OF THE INVENTION

Due to rising health care costs and increased public availability of medical information, individuals have begun to monitor their personal health independent of medical professionals. A large number of households currently include at least one health value measuring device such as a scale, blood pressure monitor, heart rate monitor, or body fat analyzer. These devices are used to measure at least one value related to an individual's overall health. For example, a scale is used to measure the body weight of a user. Body weight is often tracked to determine if weight is gained or lost over a particular time period. Likewise, weight may be used to self diagnose degrees of obesity which are known to lead to a variety of health related problems. In addition to elective health monitoring, many individuals are required to measure certain health values as a result of one or more medical conditions. For example, a diabetic must monitor his or her insulin levels to determine when it is necessary to ingest or inject supplemental insulin. Likewise, certain heart related conditions require that a patient regularly monitor his or her blood pressure.

In certain circumstances, medical professionals may be required to routinely measure one or more health values to ensure that a patient is healthy. Often, patients that require consistent measurements must be located in a hospital or medical care facility to allow medical professionals to analyze the values. For example, in certain individuals, rapid increases or decreases in blood pressure may indicate a severe medical condition, which must be treated immediately. If a patient is disabled mentally or physically it may not be practical to accurately and reliably perform these measurements. To address this problem, various companies have designed devices and systems to allow for the remote monitoring of patients. These systems allow participants to measure their values and report them to medical professional via some form of communication device. This enables the medical professional to remotely review the values to determine if further intervention is necessary.

However, the existing systems fail to overcome many of the problems encountered in pursuit of accurate, convenient medical value measurement. Participants and patients are often still required to manually measure one or more values. Participants who are mentally or physically incapacitated will not be able to utilize this type of system because of an inability to operate the measurement equipment. In addition, participants may then be required to record the measured values and transmit information to the doctor. These tasks may also be problematic for certain disabled individuals.

In addition, conventional remote health monitoring systems are directed exclusively for severely unhealthy individuals. These systems are designed exclusively to transmit information to a medical professional for review. Therefore, there is no local interpretation of the measured health values. The only information provided to the patient is the individual measurements of each particular device.

Likewise, conventional systems are prohibitively expensive. Each of the measuring devices is often packaged independently of the overall system, thereby requiring independent power sources, user interfaces, and transmission systems. Thus, a user may be required to operate two or more measurement devices in order to provide the necessary health values for proper assessment. These devices are packaged in a manner that prevents or impedes their disposal in a particular tight location, often requiring independent storage. Further, conventional monitoring systems require an operator to install the communication link between the patient and the medical monitoring professional.

Therefore, there is a need in the industry for a medical monitoring system that may be effectively utilized by both healthy and unhealthy individuals, minimizes physical space, minimizes overall cost, and is capable of reliably recording and monitoring health care value information.

### SUMMARY OF THE INVENTION

The present invention relates to systems and methods for real time diagnostic and data correlation utilizing a computer network. One embodiment of the present invention relates to a method for interfacing a health monitoring system with an on-line medical information site to provide real-time diagnostics and or updated research data correlation. The method includes measuring and recording at least two health related values, compiling the at least two health related values into a format consistent with an online medical information database, and transmitting the compiled versions of the at least two health related values to an online medical information database. The method may further include receiving real time diagnostics from a medical professional. The method may also include receiving updated medical research data corresponding to the transmitted values. An alternative embodiment of the present invention relates to a system for providing real time remote diagnostic and correlated research data. The system includes a medical monitoring system configured to measure at least two health related values, a remote medical database which is accessible by diagnostic medical professionals and which contains updated medical research data, and a communication link between the medical monitoring system and the remote medical database.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The following description of the invention can be understood in light of the Figures, which illustrate specific aspects of the invention and are a part of the specification. Together with the following description, the Figures demonstrate and explain the principles of the invention. The Figures presented in conjunction with this description are views of only particular—rather than complete—portions of the systems and methods of making and using the port system according to the invention. In the Figures, the physical dimensions may be exaggerated for clarity. The same reference numerals in different drawings represent the same element, and thus their descriptions will be omitted.

FIG. 1 illustrates a perspective view of one embodiment of a customized product in accordance with embodiments of the present invention;

FIG. 2 illustrates a flow chart of a method for measuring and recording at least two independent health values so as to produce multivariable diagnostic information in accordance with one embodiment of the present invention; and

FIG. 3 illustrates a system for performing multivariable health related diagnostics in accordance with one embodiment of the present invention.

### DETAILED DESCRIPTION OF THE INVENTION

With reference now to the drawings, the preferred embodiment of the application tool is herein described. It should be noted that the articles “a”, “an” and “the”, as used in this specification, include plural referents unless the content clearly dictates otherwise.

The present invention relates to systems and methods for real time diagnostic and data correlation utilizing a computer network. One embodiment of the present invention relates to a method for interfacing a health monitoring system with an on-line medical information site to provide real-time diagnostics and or updated research data correlation. The method includes measuring and recording at least two health related values, compiling the at least two health related values into a format consistent with an online medical information database, and transmitting the compiled versions of the at least two health related values to an online medical information database. The method may further include receiving real time diagnostics from a medical professional. The method may also include receiving updated medical research data corresponding to the transmitted values. An alternative embodiment of the present invention relates to a system for providing real time remote diagnostic and correlated research data. The system includes a medical monitoring system configured to measure at least two health related values, a remote medical database which is accessible by diagnostic medical professionals and which contains updated medical research data, and a communication link between the medical monitoring system and the remote medical database.

The following terms are defined as follows:

Multivariable diagnostic information—diagnostic information that is obtained by analyzing health related values concurrently rather than independently. Multivariable diagnostics may detect medical conditions from independent health related values even if the independent health related values appear normal when analyzed independently. Multivariable diagnostics require at least two measured health related values.

Health related values—Any type of information related to an individual's health including numerical values, images, sounds, samples, etc. Health related information includes but is not limited to height, weight, blood pressure, heart rate, insulin level, blood analysis, X-ray images, audible cough, hair sample, etc.

Health related devices—A device that is in communication with a health care monitoring system. Health related devices may measure health related data values and transmit information to the health care monitoring system. In addition, a health related device may provide a health related function such as an emergency response button. Other health related devices include but are not limited to a weight scale, height measurement, a thermometer, a digital stethoscope, a blood pressure cuff, a pulse oximeter, an ECG/rhythm strip, a glucose meter, and a first aid kit.

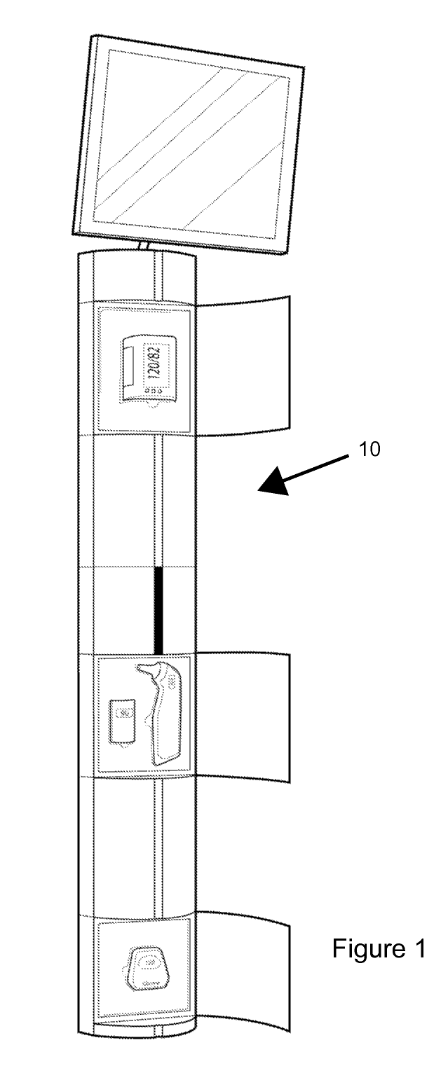

Reference is initially made to FIG. 1, which illustrates a perspective view of a medical monitoring device 10 in accordance with one component of the present invention.

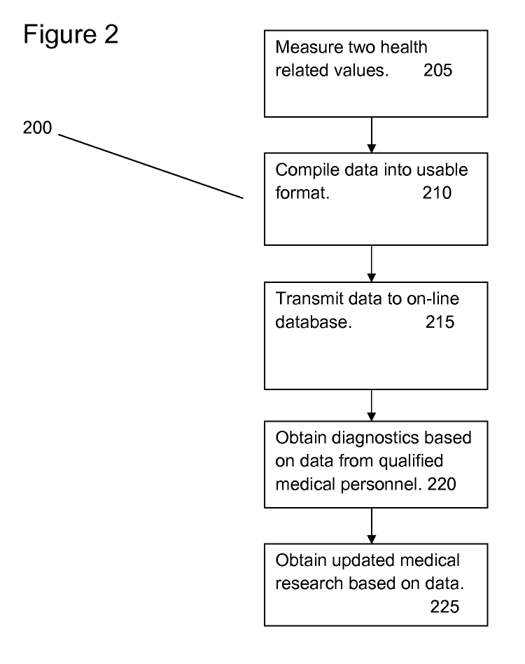

Reference is next made to FIG. 2, which illustrates a flow chart of a method for measuring and recording at least two independent health values so as to produce multivariable diagnostic information in accordance with one embodiment of the present invention, designated generally at 200. The method includes initially measuring and recording at least two health related values, act 205. The act of measuring and recording the at least two health related values may be conducted by a health monitoring device such as the one illustrated in FIG. 1. The at least two health related values are then compiled into a format consistent with an online database, act 210. Online databases are configured to receive information in a particular format, and it is therefore necessary to properly format recorded data before transmission. The compiled values are then transmitted to the online medical information database, act 215. One example of an online medical information database is WebMD.com. Real time diagnostics may then be received from one or more medical professionals based on the transmitted values, act 220. In addition, updated medical research information may also be received from the online medical information database corresponding to the transmitted values, act 225. It should be noted that, in place of an online medical database and anonymous medical personnel, a person's personal physician could be substituted in this method, thereby facilitating the communication between doctor and patient and allowing for regular data on a patient to be obtained by a doctor for monitoring an ongoing condition.

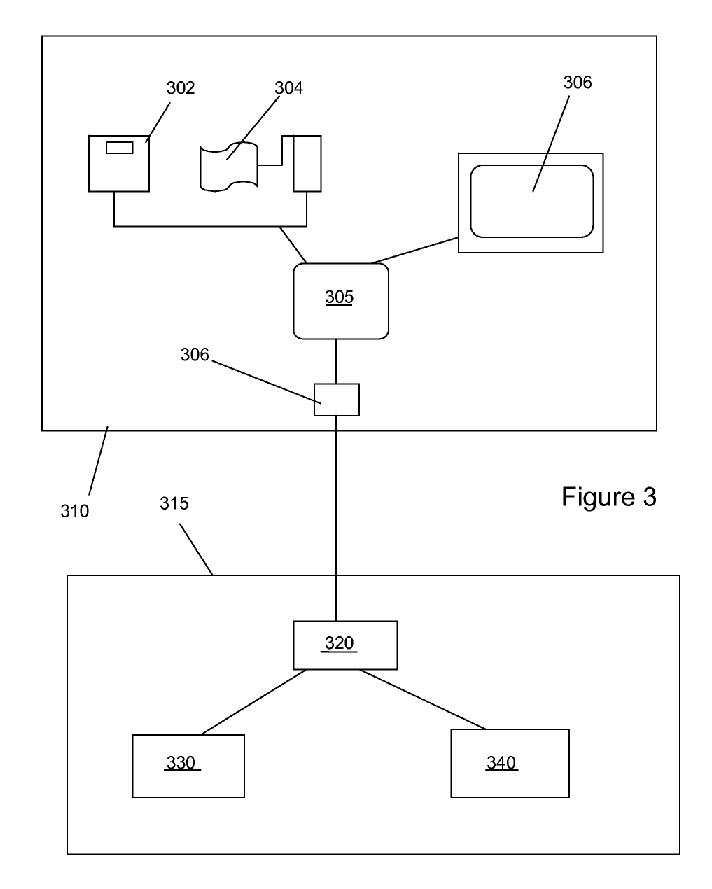

Reference is next made to FIG. 3, which illustrates a system for performing multivariable health related diagnostics in accordance with one embodiment of the present invention, designated generally at 300. The system 300 includes a medical monitoring system 310 including at least two health related measurement devices 302, 304, a communication module 308, a processor 305, and an output display 306. The medical system 300 further includes a diagnostic system 315 comprising a medical database 320, a diagnostic medical professional 330, and medical research data 340. The medical monitoring system 310, diagnostic medical professional 330, and the research data 340 are all linked to the medical database 320 via some form of communication medium such as the Internet. The medical information database 320 provides a medium upon which the diagnostic medical professionals 330 are able to analyze information from the medical monitoring system 310 in conjunction with research data 340 to produce diagnostics which may then be transmitted to the medical monitoring system 310. Likewise, research data 340 may be automatically correlated by the medical database and subsequently transmitted to the medical monitoring system 310 based on values received from the medical monitoring system 310. As noted before, any part, or the entire diagnostic system 315 could be a person's personal physician.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method for interfacing a health monitoring system with an online medical information site to provide real-time diagnostics comprising the acts of:
a. measuring and recording at least two health related values;
b. compiling the at least two health related values into a format consistent with an online medical information database;
c. transmitting the compiled versions of the at least two health related values to an online medical information database;
d. receiving real time diagnostics from one or more medical professionals, wherein the medical professionals generate the diagnostic information by viewing and analyzing the values on the online medical information database; and
e. receiving updated medical research data from the online medical database corresponding to the transmitted values.

2. The method of claim 1, wherein the online database is WebMD.

3. A system for providing real time remote diagnostic and correlated research data comprising:
a. a medical monitoring system configured to measure at least two health related values;
b. a remote medical database, which is accessible by diagnostic medical professionals, and which contains updated medical research data; and
c. a communication link between the medical monitoring system and the remote medical database.

4. The system of claim 3, wherein the medical monitoring system includes at least two measurement devices, a processor, and an output.

5. A system for providing real time remote diagnostic data comprising:
a. a medical monitoring system configured to measure at least two health related values of a patient;
b. the patient's personal physician; and
c. a communication link between the medical monitoring system and the physician.

6. The system of claim 3, wherein the medical monitoring system includes at least two measurement devices, a processor, and an output.
